(19) United States
(12) Patent Application Publication
(10) Pub. No.: US 2007/0025897 A1
(43) Pub. Date: Feb. 1, 2007

(54) Application system with recycle and related use of antimicrobial quaternary ammonium compound

(75) Inventors: Joe Rheingans, Rogers, AR (US); Kelly Beers, Fayetteville, AR (US); Chris Hawk, Little Rock, AR (US); Gary Nolen, Bella Vista, AR (US); Omar Trujillo, Rogers, AR (US); Luke Wright, Bella Vista, AR (US)

Correspondence Address: Safe Foods Corporation, 4801 North Shore Drive, North Little Rock, AR, 72118, US

(73) Assignee: Safe Foods Corporation, North Little Rock, AR (US)

(21) Appl. No.: 11/409,514

(22) Filed: Apr. 21, 2006

Related U.S. Application Data
(63) Continuation-in-part of application No. 10/535,030.
(60) Provisional application No. 60/425,679, filed on Nov. 12, 2002.
(60) Provisional application No. 60/674,230, filed on Apr. 22, 2005.

(51) Int. Cl. A61L 2/18

(52) U.S. Cl. 422/292

(57) ABSTRACT

An antimicrobial application system is disclosed, comprising an antimicrobial application unit and a recycle unit. An initial, dilute antimicrobial composition is prepared. The composition is provided to the antimicrobial application unit and applied to work pieces, such as raw poultry. After application to the work pieces, the composition is returned to the recycle tank. The concentration of the antimicrobial in the recycle tank is monitored, and additional antimicrobial is automatically added if the concentration of the antimicrobial in the composition falls below a desired amount. The composition is periodically diverted to a capture tank, and the antimicrobial is selectively removed from the composition. The removed antimicrobial and remaining composition are then disposed of in appropriate manners. The antimicrobial is preferably a quaternary ammonium compound, is more preferably an alkylpyridinium chloride, and is most preferably cetylpyridinium chloride.

This application claims priority as a continuation-in-part of U.S. patent application Ser. No. 10/535,030, filed May 11, 2005, which was the National Stage of International Application No. PCT/US03/35933, filed Nov. 12, 2003, and which claims the benefit of U.S. Provisional Application Ser. No. 60/425,679, filed Nov. 12, 2002.

This application also claims the benefit of U.S. Provisional Patent Application Ser. No. 60/674,230, filed Apr. 22, 2005.

U.S. patent application Ser. No. 10/535,030, International Application No. PCT/US03/35933, and U.S. Provisional Patent Application Nos. 60/425,679 and 60/674,230 are incorporated herein by reference.

### BACKGROUND OF THE INVENTION

This invention relates to an antimicrobial application system, and more particularly to an antimicrobial application system with recycle features for use in connection with food products and surfaces and other items associated with food processing.

Antimicrobial application systems, including spray cabinets are known in the art. U.S. Pat. No. 6,742,720, issued Jun. 1, 2004, discusses a number of such systems and highlights a number of the advantages and disadvantages of these systems. The disclosure of U.S. Pat. No. 6,742,720 is incorporated herein by reference. The spray application systems disclosed in that patent offer a number of advantages over earlier systems, as discussed in more detail in that patent. Still, the present inventors have further refined and built upon those systems to offer alternate embodiments offering additional flexibility. For example, it may be desirable to recycle the antimicrobial that is applied to the work pieces. Adding equipment and steps to allow for recycling adds to the cost and complexity of a system, so it will not always be preferred. Still, using recycling reduces consumption of the antimicrobial and water and reduces the amount of waste material in need of disposal. This may be desirable for any number of reasons such as environmental concerns, raw material costs, raw material storage limitations, disposal costs, and regulatory issues involving disposal of wastewater and some antimicrobials. Accordingly, under many circumstances, it will be desirable to recycle the antimicrobial for multiple applications to work pieces to be treated.

Recycling of liquids applied to some types of work pieces in a process line is generally known in the art. Still, recycling liquids in connection with food processing and items associated with food processing presents a number of special issues and concerns, particularly concerning adulteration, contamination, and cross-contamination. These concerns typically argue against recycling or lead to the use of slow, cumbersome, undesirable extra steps and extra equipment that add to the cost and complexity of a system. One such complex system is disclosed in U.S. Pat. No. 6,348,227, issued to Caracciolo, Jr. in 2002, the disclosure of which is incorporated herein by reference.

### SUMMARY OF THE INVENTION

It is therefore an object of the present invention to provide an antimicrobial application system that provides for the safe, effective, and cost efficient recycling of antimicrobial in connection with food processing and items associated with food processing.

It is a further object of the present invention to provide a system of the above type that reduces raw material consumption without sacrificing safety.

It is a still further object of the present invention to provide a system of the above type that provides for periodic, batch style separation and disposal of spent antimicrobial.

It is a still further object of the present invention to provide a system of the above type which automatically monitors and maintains a desired composition of the antimicrobial composition to be recycled.

It is a still further object of the present invention to provide a system of the above type which provides for improved recapture and return of an antimicrobial composition applied to work pieces.

It is a still further object of the present invention to provide a system of the above type which automatically compensates for additional liquids passing from wetted work pieces to the recycled antimicrobial composition.

It is a still further object of the present invention to provide a system of the above type which is capable of providing continuous, real-time monitoring and control of the composition of an antimicrobial composition.

It is a still further object of the present invention to provide a system of the above type which reduces waste leaving the system and waste disposal costs associated therewith.

It is a still further object of the present invention to provide a system of the above type which provides a safe waste stream that may be safely drained into a wastewater system.

It is a still further object of the present invention to provide a system of the above type that increases the flexibility and advantages of the spray application systems and spray cabinets disclosed in U.S. Pat. No. 6,742,720 and in PCT Application Serial Number PCT/US03/35933.

It is a still further object of the present invention to provide a system of the above type that may effectively apply, capture, and reapply a solution that is prone to foaming.

It is a still further object of the present invention to provide a system of the above type that provides increased flexibility in positioning and utilization of spray nozzles.

It is a still further object of the present invention to provide a system of the above type that handles large fluctuations in processing requirements.

It is a still further object of the present invention to provide a system of the above type that is relatively easy to install, clean, and maintain.

It is a still further object of the present invention to provide a system of the above type that provides a simple, reliable method of monitoring and controlling the composition of an antimicrobial to be recycled, even when the antimicrobial contains impurities.

Toward the fulfillment of these and other objects and advantages, the antimicrobial application system of the present invention comprises an antimicrobial application unit and a recycle unit. An initial, dilute antimicrobial composition is prepared and the concentration of the antimicrobial is controlled automatically. The composition is provided to the antimicrobial application unit and applied to work pieces, such as raw poultry carcasses. After application to the work pieces, the composition is returned to the recycle tank of the recycle unit. The concentration of the antimicrobial returning to the recycle tank is monitored, and additional antimicrobial is automatically added if the concentration of the antimicrobial in the composition falls below a desired amount. The composition is periodically diverted to a capture tank, and the antimicrobial is selectively removed from the composition. The removed antimicrobial and remaining composition are then disposed of in appropriate manners. The antimicrobial is preferably a quaternary ammonium compound, is more preferably an alkylpyridinium chloride, and is most preferably cetylpyridinium chloride.

### BRIEF DESCRIPTION OF THE DRAWINGS

The above brief description, as well as further objects, features and advantages of the present invention will be more fully appreciated by reference to the following detailed description of the presently preferred but nonetheless illustrative embodiments in accordance with the present invention when taken in conjunction with the accompanying drawings, wherein:

FIG. 1 is a schematic view of an antimicrobial application system of the present invention;

FIG. 2 is a side elevation view of an adjustable bracket used in connection with the present invention;

FIG. 3 is a partially exploded, side elevation view of an application unit of the present invention;

FIG. 4 is a schematic view of an alternate embodiment of an application unit of the present invention;

FIG. 5 is a side elevation view of a recycle skid of the present invention;

FIG. 6 is another side elevation view of the recycle skid of the present invention; and

FIG. 7 is a side elevation view of a foam dissipating recycle tank insert.

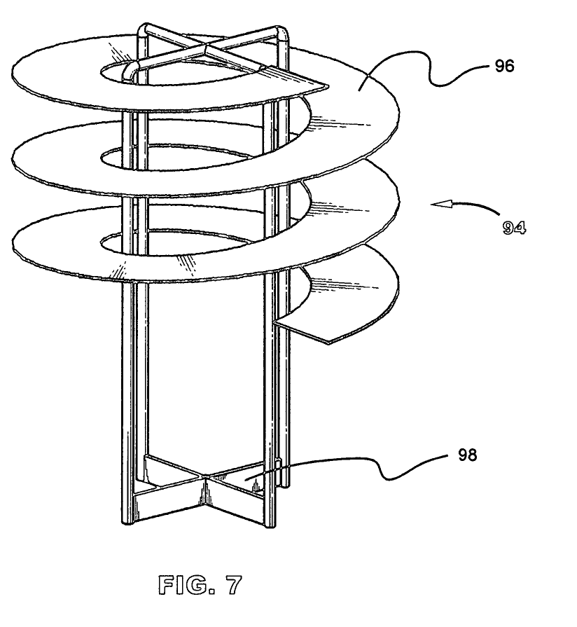

### DETAILED DESCRIPTION OF THE PREFERRED EMBODIMENTS

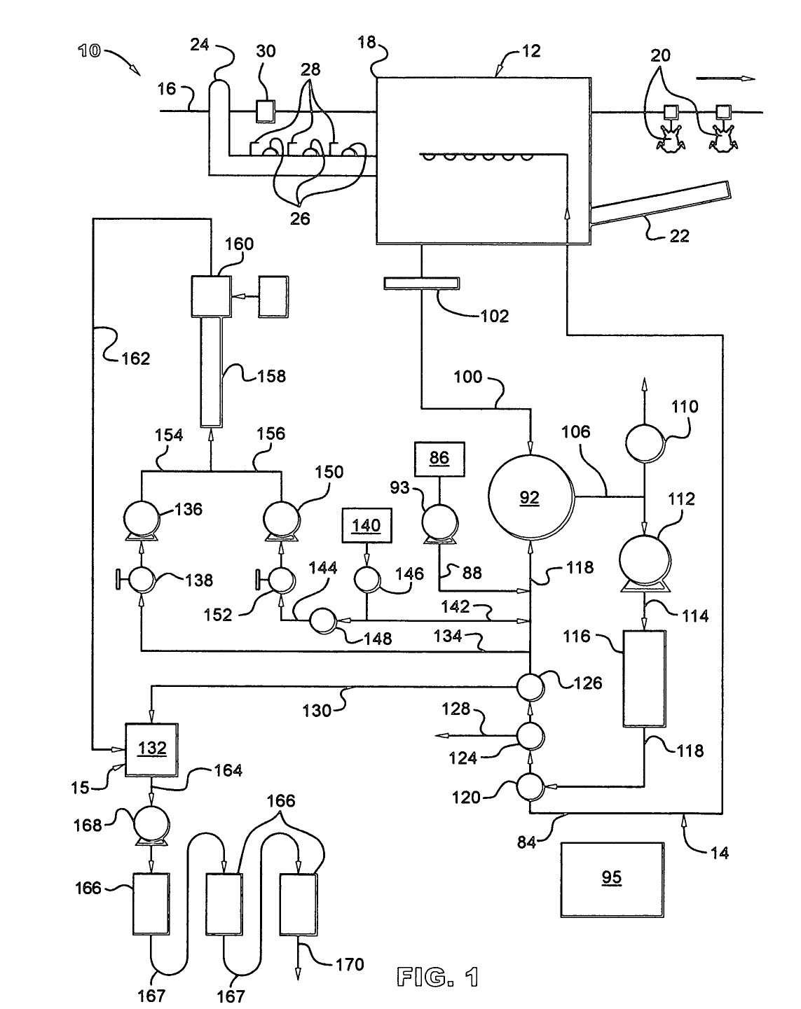

Referring to FIG. 1, the reference numeral 10 refers in general to an antimicrobial application system of the present invention. The antimicrobial application system 10 of the present invention generally comprises an antimicrobial application unit 12 and a recycle unit 14, and may include a capture unit 15.

The antimicrobial application unit 12 may take any number of configurations. In the preferred embodiment, the antimicrobial application unit 12 takes the general form of one of the embodiments of a spray application system as disclosed in U.S. Pat. No. 6,742,720 or in PCT Application Serial Number PCT/US03/35933. One possible exception is that the liquid barriers described in U.S. Pat. No. 6,742,720 are not used in the preferred embodiment of the present invention. A conveyor 16 passes through a housing 18 for moving work pieces 20, such as raw poultry carcasses, through the housing 18. As described in more detail below, a drip tray or pan 22 extends downstream of the housing 18, disposed below the conveyor 16 and the work pieces 20 carried thereby.

A rigid member 24, such as stainless steel tubing, is affixed to the housing 18, preferably at an upstream end of the housing 18. The rigid member 24 has parallel arms that are aligned on opposite sides of the conveyor line 16. A series of matching openings are provided in each arm for housing counters or sensors 26. Protective lenses provide watertight seals, preferably NEMA 4 seals, to protect the counters from damage that might otherwise occur under the harsh washdown conditions to which the systems 10 are routinely subjected. Nozzles 28, preferably connected to a source of pressurized air, are disposed near each of the sensors 26 for reasons to be discussed below. Three counters 26 are preferably provided in series. As best seen in FIG. 1, the arms are disposed so that the counters are aligned to detect the presence or absence of work pieces 20.

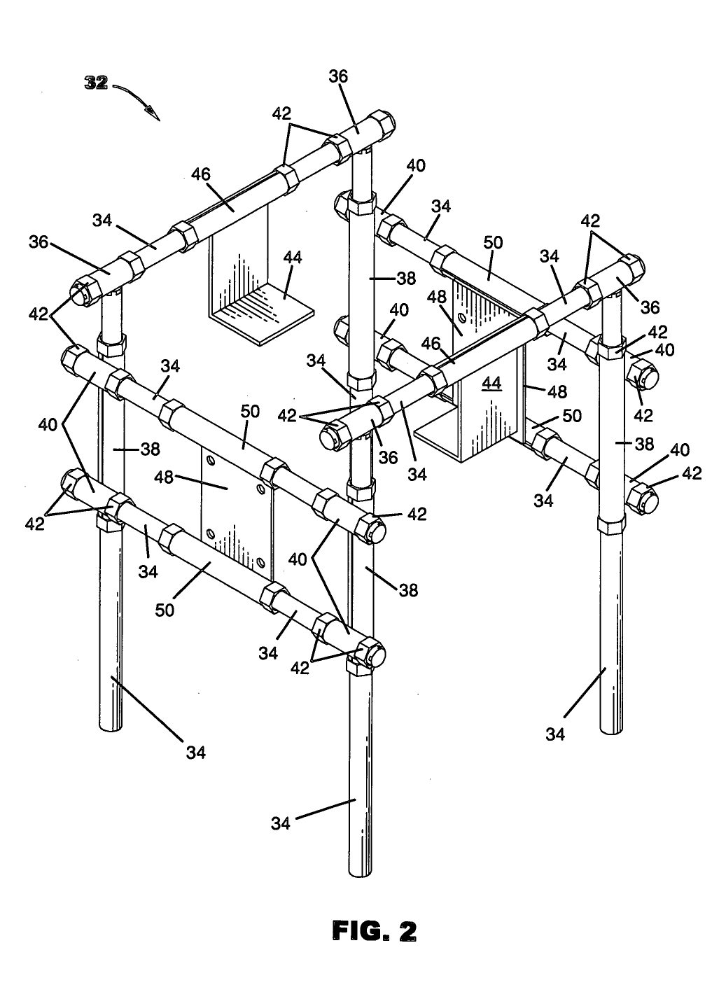

A proximity switch 30 is mounted on the conveyor line using an adjustable bracket 32. Referring to FIG. 2, a frame is formed by threaded rods 34, sleeves 36, 38, and 40, and nuts 42. Generally L-shaped brackets 44 are affixed to sleeves 46, and plates 48 are affixed to sleeves 50. Proximity switches 30 are affixed to plates 48, and brackets 44 are affixed to the top of the conveyor or shackle line 16, such as by welding. Sleeves 36 and 46 allow for horizontal adjustments of the proximity switches 30 toward or away from the line 16, sleeves 38 allow for vertical adjustments of the proximity switches 30, and sleeves 50 allow for horizontal adjustments of the proximity switches 30 longitudinally along the line 16.

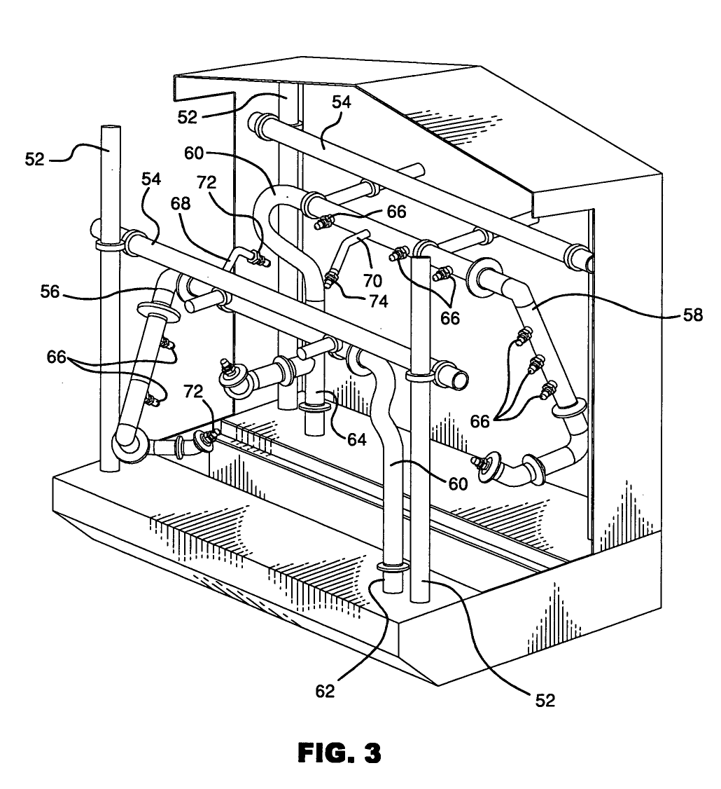

Referring to FIG. 3, vertical members 52 are affixed within the housing 18. Horizontal members 54 are adjustably secured to members 52, allowing vertical adjustments of members 54. Breast-side manifold 56 and back-side manifold 58 are adjustably secured to members 54, allowing horizontal adjustments of the manifolds 56 and 58 to move them toward or away from the line 16 and work pieces 20. Flexible tubing, such as reinforced clear hose 60 connects inlet pipes 62 and 64 with manifolds 56 and 58. Spray nozzles 66 are positioned as desired along the manifolds and inlet pipes. Extensions 68 and 70 are used to position nozzles 72 and 74 for better spray coverage of the vent areas of poultry carcasses 20 passing on shackles through the housing 18. Most of the nozzles 66 and 74 are preferably cone spray nozzles that form generally cone shaped patterns having angles of approximately 65°. This spray pattern provides a good balance of spray coverage and impact pressure for most of the areas to be treated. Breast-side nozzle 72, that is positioned on extension 68 for treating the vent areas of poultry carcasses 20, preferably has a tighter, more focused cone-shaped pattern, the cone preferably having an angle of less than or equal to approximately 30° and having an angle of greater than or equal to approximately 15°.

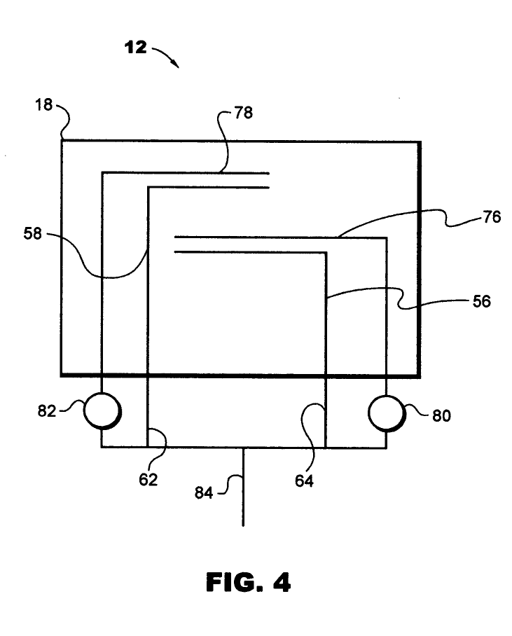

An alternate embodiment of an antimicrobial application unit 12 is shown in FIG. 4. For reasons discussed below, additional manifolds 76 and 78 are provided, affixed to manifolds 56 and 58 respectively. Valves 80 and 82 allow flow through manifolds 76 and 78 to be turned on and off as desired. Accordingly, flow from feed pipe 84 may be directed not only to manifolds 56 and 58 but also to manifold 76 and/or 78 as desired. It is of course understood that manifolds 76 and 78 may be affixed and disposed within the housing 18 in any number of ways, that more or fewer manifolds may be used, and that any number of different routing schemes may be used to supply liquid to and to cut off liquid from the manifolds.

It is of course understood that the antimicrobial application unit 12 is not limited to the embodiments discussed above and in the references incorporated herein or even to spray application systems in general. The antimicrobial application unit 12 may apply a composition such as an antimicrobial composition to any number of different kinds and types of work pieces 20 in any number of different ways. Methods of application used by such an application unit may include but are not limited to spraying, misting, fogging, immersing, pouring, dripping, and combinations thereof. It is understood that the system may be used to treat a wide variety of different work pieces 20, including but not limited to meat, poultry, fish, fresh and salt water seafood, fruits, vegetables, other foodstuffs, animals, food packaging, and items and surfaces related to food or food processing. It is also understood that the work pieces 20 may be live, dead, raw, hide-on, carcass, pieces, cooked, prepared, processed, partially processed, ready to eat, or ready to cook. It is further understood that the system 10 may be used to treat work pieces 20 completely unrelated to food or food processing items.

The recycle unit 14 dilutes a concentrated antimicrobial composition or solution to obtain a dilute antimicrobial composition or solution and provides the dilute antimicrobial solution to the antimicrobial application unit 12. An antimicrobial source 86, such as a supply tank, is connected via antimicrobial supply line or conduit 88 to a port 90 of the recycle tank 92. A chemical feed pump 93 is disposed in antimicrobial supply line 88. The pump is operably connected to a controller 95 for reasons to be described below. The antimicrobial preferably comprises a quaternary ammonium compound, more preferably comprises an alkylpyridinium chloride, and most preferably comprises cetylpyridinium chloride (CPC). More particularly, the antimicrobial solution preferably comprises a concentrated solution of a quaternary ammonium compound as described in U.S. Pat. No. 6,864,269, the disclosure of which is incorporated herein by reference. The concentrated solution preferably comprises an antimicrobial and a solubility enhancing agent, and the solubility enhancing agent preferably comprises propylene glycol. The quaternary ammonium compound is preferably present in the concentrated solution in a weight percent of approximately 40%, and the solubility enhancing agent is preferably present in the concentrated solution in a weight percent of approximately 60%. It is of course understood that any number of different antimicrobials and solubility enhancing agents may be used, and that the concentrated and dilute solutions may have any number of different components and compositions, including but not limited to the components and compositions of the concentrated and dilute solutions disclosed in U.S. Pat. No. 6,864,269. It is also understood that the concentrated solution may contain any number of additional components, including but not limited to water, preservatives, anti-oxidants, anti-foaming agents, such as dodecanol, and the like. Concerns of adulteration, contamination, or cross-contamination are eliminated or alleviated because of the broad-spectrum efficacy of the preferred antimicrobial solutions.

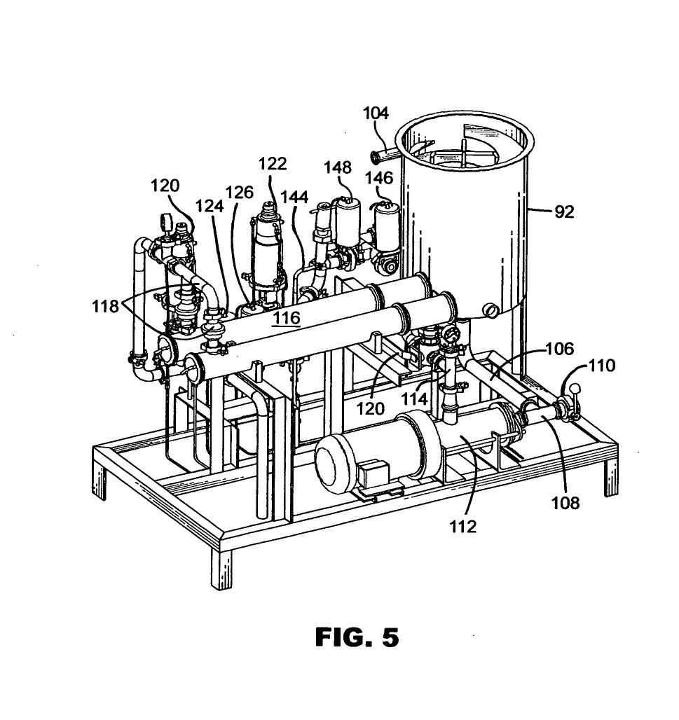

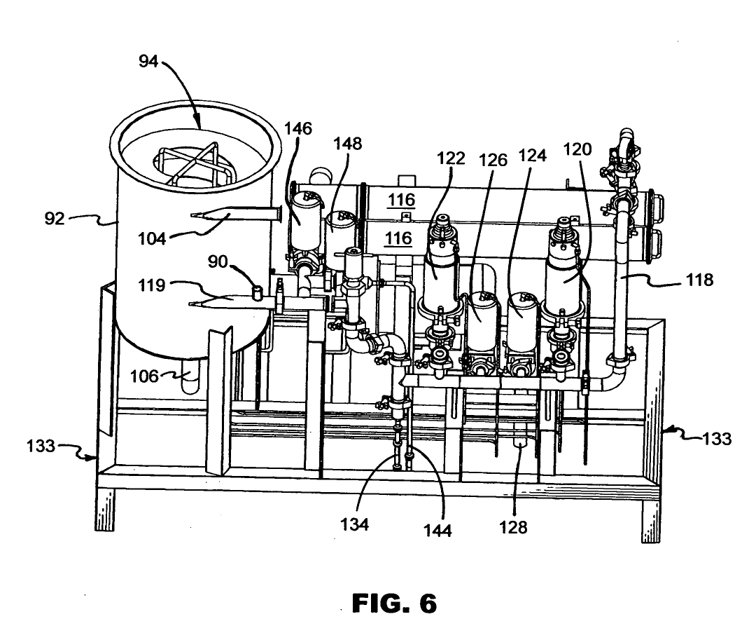

One or more recycle tanks 92 are provided. A removable insert 94 is disposed within the tank. The insert has a flight 96 disposed in an upper portion of the tank and baffle members 98 disposed in a lower portion of the tank. The flight 96 is preferably a helical or spiral member and may have an inner flange extending along its inner edge to reduce the amount of liquid that spills over the inner edge of the flight. The upper portion of the flight 96 preferably extends up to approximately 80% of the height of the tank 92, and the lower portion of the flight 96 preferably extends down to approximately 40% of the height of the tank 92. Positioning stops, flanges, or members may be disposed within the tank 92 to retain the insert 94 in a desired orientation, but the insert 94 is easily removed for cleaning and is preferably not affixed to the tank 92. There is a gap of approximately ¼ to approximately 3/16 between the outer edge of the insert flight 96 and the inner surface of the tank 92. Although the recycle tank 92 may include an impeller or some other stirring or agitation means, no such stirring or agitation means is used in the preferred embodiment. Although omitted from FIGS. 5 and 6 for purposes of clarity and presentation, it is understood that recycle tank 92 has a removable, sealed cover at the top.

A return line or conduit 100 extends between the housing 18 and the recycle tank 92 for passing liquid from the housing 18 to the tank 92. Multiple return lines 100 may be used to connect multiple antimicrobial application units 12 to the recycle tank 92. A filter 102 is disposed in the housing 18 or in the return line 100. The filter 102 is preferably disposed within the housing 18 and is preferably a wire mesh filter, such as a 100 mesh filter, sized to capture visible particulate matter in the effluent from the antimicrobial application unit 12. Visible particulate matter in the effluent will typically be minimal because of upstream washing that will typically be performed on the work pieces 20. The return line 100 preferably returns the liquid to inlet 104 that is positioned at approximately 80% of the height of the tank 92 and is preferably aligned to provide for substantially tangential feeding of the return liquid.

If the antimicrobial has a tendency to foam, the return line 100 is disposed and routed to provide for a gentle slope between the housing 18 and tank 92. It is preferred that the return line 100 not experience large or sudden drops in elevation. A substantial portion of the return line is disposed to provide a drop that is preferably less than or equal to approximately 1″ per foot of line, that is more preferably less than or equal to approximately ¼ per foot of line and that is most preferably less than or equal to approximately ⅛ per foot of line. This gentle sloping preferably extends over a substantial portion of the line 100, more preferably extends over at least approximately 80% of the length of the line 100, and most preferably extends over substantially all of the line 100.

Discharge line 106 exits from the bottom of tank 92 and extends to tee 108. Discharge valve 110 is provided for use during cleaning and draining. Tee 108 is connected to pump 112. Line 114 extends between pump 112 and filters 116, entering the filters 116 from below. Line 118 exits filters 116 from above to reduce or eliminate the chances that debris will fall into the post-filter line 118 during cleaning. The first and second filters 116 are associated with each tank 92 and are disposed to provide for parallel flow. Valve 120 or other means are provided for selectively directing liquid through either the first filter or the second filter. This allows the system 10 to continue operating while one of the filters 116 is being cleaned, replaced, or repaired. Line 118 extends between the filters 116 and the recirculation inlet 119 of the recycle tank 92. Inlet 119 is positioned at approximately 40% of the height of the tank 92 and is preferably aligned to provide for substantially tangential feeding of the liquid being recirculated. Inlet 119 is also preferably disposed to reintroduce the recirculation liquid below the liquid level in the tank 92, below the flight 96 of insert 94, and above the baffles 98 of insert 94.

Modulating valves 120 and 122 and butterfly valves 124 and 126 are disposed along line 118. Feed line 84 extends from modulating valve 120 to the housing 18 to supply manifolds 56, 58, 76, and 78 and their associated nozzles 66, 72, 74. Discharge line 128 extends from butterfly valve 124 for use during cleaning and draining of the system 10. A purge or capture line 130 passes from butterfly valve 126 to the capture tank 132. The embodiment depicted in FIG. 1 is for use in connection with a single line 16 application, whereas the embodiment depicted in FIGS. 5 and 6 is for use in connection with a two line 16 application. As seen in FIGS. 5 and 6, a second modulating valve 122 is provided for supplying liquid to manifolds and nozzles in a second housing. It is of course understood that separate modulating valves may be used for each manifold or that a single modulating valve may be used to supply liquid to manifolds disposed in more than one housing.

The recycle tank 92, pump 112, filters 116, line 118, modulating valves 120 and 122, butterfly valves 124 and 126, and a number of other components are preferably affixed to a recycle skid assembly 133 formed from angle irons arranged to form a frame. The skid 133 makes installation faster and easier and offers a number of advantages. If the antimicrobial used is subject to foaming, the skid 133 is preferably configured to support the recycle tank 92 in an elevated position to reduce the vertical distance that liquid exiting the housing 18 must drop before entering the tank 92. If the antimicrobial used is subject to foaming, the skid supports the tank 92 so that the vertical drop needed to pass the liquid from the bottom of the housing 18 to inlet 104 is preferably less than or equal to approximately 5 feet, is more preferably less than or equal to approximately 3 feet, and is most preferably less than or equal to approximately 2 feet.

A diverting line 134 is also disposed in the line 118. The diverting line 134 is connected to a metering or dilution pump 136 and has a pressure regulator 138 disposed therein. A source of potable water 140, such as tap water, is provided and is connected to line 118 via line 142. A diverting line 144 is also disposed in water supply line 142. Butterfly valves 146 and 148 control the flow of water to lines 142 and 144. The diverting line 144 is connected to a metering or dilution pump 150 and has a pressure regulator 152 disposed therein. Dilution pumps 136 and 150 are electrically interlocked to provide for matched, stroke for stroke pumping action. Lines 154 and 156 exit the dilution pumps 136 and 150 and then connect and route liquids from the dilution pumps 136 and 150 to a static mixer 158. The static mixer is preferably an inline, auger style static mixer.

A sensor 160 is disposed at the discharge end of the static mixer 158. In the preferred embodiment, the sensor 160 is a multi-channel ultraviolet light spectrophotometer or UV spec sensor. Of course it is understood that any number of different types of sensors 160 may be used, including but not limited to infrared, visible light, or ultraviolet sensors. The sensor 160 is capable of detecting the concentration of the antimicrobial in the solution exiting the static mixer 158. The controller 95 operably connects the sensor 160 to the chemical feed pump 93. The controller 95 is capable of receiving a signal from the sensor 160 and sending a corresponding on/off signal to the chemical feed pump 93. A discharge line 162 passes from the sensor 160 to the capture or purge tank 132.

A drain line 164 passes from the capture tank 132 to one or more antimicrobial separation tanks or drums 166. A pump 168 is provided for passing liquid from the capture tank 132 to the drums 166. The drums 166 preferably include one or more filters, such as disposable carbon filters, that selectively remove the antimicrobial from the composition. A disposal line 170 exits the last drum 166 for disposing of water and any other components remaining after the antimicrobial is selectively removed. It is understood that a separation unit 15 may or may not be used and that any number of different separation methods may be used. It is also understood that the filters may be disposable or reusable. The central control unit 95 is used to control the entire system 10.

In operation, a dilute antimicrobial solution will typically be prepared and used for one spray cycle that will typically last for one day. The dilute antimicrobial solution will then be discarded, disposed of, or removed from the system 10 for further processing. It is of course understood that the spray cycle may be of any number of different durations. It is also understood that the system 10 may be operated in batch mode, in steady-state mode, or in any number of different types or combinations of modes of operation. A new spray cycle will typically begin each morning with an empty and clean recycle tank 92 and an empty and clean capture tank 132. Before the antimicrobial application unit 12 is activated, and before the system pump 112 is turned on, the dilute antimicrobial solution is prepared. In that regard, a desired amount of tap water is fed to the recycle tank 92. The recycle tank 92 is preferably filled to approximately one third to approximately one half of its capacity with potable water and is more preferably filled to a level of at least 40% of the height of the tank 92. The concentrate pump 93 is activated to feed the concentrated antimicrobial composition to the tank 92 until a predetermined amount of the concentrate composition is provided. The concentrate composition combines with the water in the recycle tank 92 to form a dilute solution of the desired concentration. The desired ranges of the concentration of antimicrobial in dilute solution include but are not limited to the concentration ranges of the antimicrobial in the dilute solutions disclosed in U.S. Pat. Nos. 5,855,940, 6,039,992, and 6,864,269, the disclosures of which are incorporated herein by reference.

Once the desired concentration is obtained in the recycle tank 92, the system pump 112 is activated, and the dilute solution is supplied to the antimicrobial application unit 12. The dilute solution provided to the antimicrobial application unit 12 is not potable. Still, contamination or cross-contamination of the work pieces 20 is not a concern because of the safety and broad spectrum efficacy of the dilute antimicrobial solution used. The recycle unit 14 supplies the dilute antimicrobial solution to the antimicrobial application unit or units 12 at any number of different flow rates and pressures. These flow rates and pressures may include, but are not limited to, the flow rates and pressures discussed in U.S. Pat. No. 6,742,720, the disclosure of which is incorporated herein by reference. Line 118 routes a portion of the dilute composition to a lower portion of the tank 92 through inlet 119 so that it does not pass through the manifolds 56, 58, 76, 78 and nozzles 66, 72, and 74 and is not applied to the work pieces 20. The ratio of dilute composition passing through the bypass inlet 119 versus passing to the feed line 84 will typically be greater than or equal to approximately 1:1 and will more typically be greater than or equal to approximately 2:1. The dilute composition passing through the inlet 119 provides for improved mixing of the dilute solution and any concentrate composition that might be added. The use of the return line 118 and inlet 119 also provides greater flexibility in providing dilute solution to manifolds 56, 58, 76, and 78 at or within fluctuating ranges of flow rates and pressures. The use of the return line 118 and inlet 119 also makes it easier to continue to provide dilute solution to the manifolds 56, 58, 76, and 78 at consistent pressures and flow rates as additional spray application units 12 or manifolds are brought online or taken offline and regardless of the number of spray application units 12 that are online.

Once the recycle unit 14 is supplying the dilute antimicrobial solution to the antimicrobial application unit 12, the work pieces 20 to be processed, such as raw poultry, are moved by the conveyor 16, through the housing 18. It will often be desirable to apply an antimicrobial or other active ingredient to the work pieces 20 within a desired range of application rates. For example, it may be desirable to apply CPC to raw poultry carcasses at a level that is less than or equal to approximately 0.3 g per pound of raw poultry carcass. To stay within the desired range while treating raw poultry carcasses 20 passing on a conveyor line 16 through a housing 18 will require dealing with factors such as the weight of the poultry carcasses 20, the speed of the line 16, the concentration of the dilute solution, and the flow rate through the nozzles 66, 72, and 74. The bird weight will typically be entered manually using an average weight of the birds to be processed. It is of course understood that the birds may be weighed while on the line 16 and the weight used by the controller 95 may be entered manually or automatically and may be an average weight or a real-time, actual weight.

The speed of the line 16 is measured by the proximity switch 30, and this information is provided to the controller 95. The proximity switch 30 will typically determine the rate at which shackles pass, and the controller can determine line speed by combining this information with known information about shackle spacing. It is of course understood that information on line speed may be entered and updated in any number of ways, manually and automatically.

It may also be desirable to have an accurate count of the actual number of work pieces 20 that are treated, such as for billing purposes. Sensors 26 are used for this purpose. The use of three counters 26 provides redundancy and increases accuracy. In that regard, the counters 26 are operably connected to the controller 95, and the counts taken by the three counters 26 are continuously compared. If one counter 96 provides a reading or count that differs from that provided by the other two, the controller 95 will typically be programmed to disregard the reading of the inconsistent counter 96 and to rely instead upon the readings of the other two counters. A counting error may sometimes be caused by debris or liquid on a lens of a counter 26. Accordingly, if one counter 96 provides a reading or count that differs from that provided by the other two, the controller 95 will typically be programmed to provide a blast of air to the lens of the appropriate counter 26, using the associated nozzle 28. If a particular counter 26 routinely provides a reading or count that differs from that provided by the other two, the particular counter will be pulled offline or disregarded, until it can be examined, repaired, replaced, or cleaned as needed. The logic and interpretation of the different readings may of course be modified in any number of ways.

As the work pieces 20 pass through the housing 18, the dilute antimicrobial solution is applied to the work pieces 20, such as by spraying. Most of the nozzles 66 will preferably provide a cone shape pattern having spray angles of approximately 65°. Adjacent nozzles 66 along a manifold 56, 58, 76, or 78 should be directed toward different portions of the work pieces 20 to avoid a sheeting action in which the spray from the second such nozzle runs off without adequate interaction with the work piece 20. Nozzle 72 on the breast-side manifold 56 preferably has a tighter, more focused cone-shaped pattern, the cone preferably having an angle of less than or equal to approximately 30° and having an angle of greater than or equal to approximately 15°. The more focused pattern allows more of the antimicrobial from nozzle 72 to pass through the vent of a poultry carcass. It is also believed that the increased impact pressure of a tighter spray pattern leads to increased splashing of the antimicrobial within the cavity and better coverage of upper areas of the cavity, such as the hard to reach undersides of flaps of skin and tissue often found around the vent area. For many of the same reasons, it may also be desirable to use the same type of nozzle 72 for treating the neck area of a carcass from below. The members 54 are adjusted vertically and manifolds 56 and 58 are adjusted laterally or horizontally to better position the nozzles 66, 72, and 74 depending upon the size and positioning of the work pieces 20. This is particularly useful in positioning nozzles 72 and 74 for better coverage of the vent, cavity, and neck areas.

In the alternate embodiment depicted in FIG. 4, manifolds 76 and 78 may be brought online or taken offline to provide greater flexibility in handling large deviations in flow rates. This, in turn, provides greater flexibility in handling large deviations in line 16 speeds, bird 20 weights, and the like. The system will typically be designed to use all manifolds 56, 58, 76, and 78 during the operating conditions that are most often expected. Accordingly, valves 80 and 82 will remain open during most normal operating conditions. Nozzles 66, 72, and 74 typically require a minimum amount of line pressure to provide effective spray patterns. For example, if line pressure drops below approximately 10 psi, the nozzles 66, 72, and 74 will typically provide a poor spray pattern. This is one of the factors that places limits on how much flow rates may be adjusted downwardly without adversely affecting the treatment of the work pieces 20. If there is a significant drop in line 16 speed or in bird 20 weight, it may become desirable to significantly reduce the flow rate going to the manifolds 56, 58, 76, and 78. To accomplish this without adversely affecting the spray pattern, the present system allows multi-staging of the manifolds to selectively take one or two of the manifolds offline. It will typically be desirable to first close valve 82 to take back-side manifold 78 offline. If a further reduction is needed, valve 80 may then be closed to take breast-side manifold 76 offline. Key nozzles 72 and 74 should be located on manifolds 56 and 58 so that they remain online at all times. When line 16 speed or bird 20 weights are increased so that the manifolds may be effectively brought back online, valves 80 and 82 are opened as appropriate.

The length of the drip tray 22 is selected so that it will catch drops from work pieces 20 exiting the housing 18 for approximately 1 minute after the work pieces 20 exit the housing 18. This enhances the recovery of the dilute antimicrobial solution and reduces downstream losses. Although not preferred, liquid barriers such as water spray curtains may be used in the housing 18. Also, the work pieces 20 may be wet from upstream washing, so additional water may enter the line 100, decreasing the concentration of the antimicrobial in the dilute solution. The portion of the dilute antimicrobial solution that does not adhere to the work pieces 20, passes through filter 102, collects in a drain, and is returned via return line 100 to the recycling tank for reuse. The gentle sloping of the return line 100 reduces foaming of the antimicrobial. The tangential feed of inlet 104 and the flight 96 of insert 94 also reduce foaming of the antimicrobial as the dilute solution accumulates in the recycle tank 92. Baffles 98 interfere with swirling of the liquid within the tank 92 and help to prevent or inhibit the formation of a vortex at or near the bottom of the tank 92. This helps to prevent air from getting into line 106 and therefore into pump 112, which would be highly undesirable.

The dilute solution leaves the tank 92 via line 106, passes through pump 112, and filter 116 to line 118. Modulating valve 120 directs the desired portion through feed line 84 back to the housing 18. The remaining portion passes through to butterfly valve 124. Butterfly valve 124 is closed during operation and is only opened as needed to clean or drain the system via line 128. The dilute solution then passes the butterfly valve 126. Butterfly valve 126 is closed during operation and is only opened as needed to clean or drain the system via line 130, which passes to capture tank 132. If additional lines 16 are being treated, additional modulating valve 122 may be disposed in line 118 as needed to divert flow as appropriate. A tee in line 118 directs some of the dilute solution into line 134 so that the concentration of antimicrobial in the solution may be monitored. The remaining portion of the dilute solution is returned to the recycle tank 92 via port 119. Port 119 preferably returns the dilute solution below the level of liquids in the recycle tank 92, below the flight 96 of the insert 94 and above the baffles 98 of the insert 94. Additional water may be provided to the system as needed via line 142 and additional concentrate antimicrobial solution may be provided to the system as needed via line 88 and port 90. Valve 146 controls the introduction of additional water to line 142, and valve 148 controls the introduction of additional water to line 144.

The system measures the concentration of the antimicrobial in the dilute solution periodically and makes adjustments as needed, such as by introducing additional concentrate solution or water or by adjusting the rate of addition of concentrate solution or water. The pressure regulators 138 and 152 preferably regulate the pressure in the lines feeding pumps 136 and 150 to a pressure lower than the pressures in lines 134 and 144 and preferably regulate the pressure in those lines down to approximately 15 psig. The dilution pumps 136 and 150 are electrically interlocked to provide for matched, stroke for stroke pumping action. The dilution pumps 136 and 150 are also sized to provide for a desired, fixed dilution ratio. The dilution ratio is preferably less than or equal to approximately 1 part dilute composition to 1 part water, is more preferably less than or equal to approximately 1 part dilute composition to 30 parts water, and is most preferably less than or equal to approximately 1 part dilute composition to 60 parts water.

A preferred sensor 160, such as a spectrophotometer, is typically used to measure very low concentrations of a component in a composition. It is therefore important to provide a liquid that has not only a relatively uniform composition but also a very low concentration of the antimicrobial or component to be measured. Often, it will not be practical or feasible to obtain accurate, reliable readings for the antimicrobial at the concentration ranges typically found in the recycle tank 92. Diluting the composition before taking a concentration reading will offer greater flexibility in the selection of a sensor 160 for monitoring the concentration of the antimicrobial. Samples of the composition exiting the recycle tank 92 are therefore taken and further diluted, to yield further diluted compositions in which the antimicrobial is present within a concentration range that is readily and accurately measured by the sensor 160. The dilution ratio of the dilution pumps 136 and 150 is selected to provide the desired degree of dilution, such as within the ranges discussed above. The pumps 136 and 150 are set on a timer to take samples at a set interval, such as every six minutes, each sample being taken for a set duration of time. It is understood that the concentration may be monitored at any number of different intervals and for any number of different durations and that the concentration may be continuously monitored. The electrically interlocked pumps 136 and 150 provide the dilute composition and water in the desired fixed ratio to further dilute the dilute composition. Using electrically interlocked pumps at a desired, fixed dilution ratio simplifies controls needed to operate the system 10. It is of course understood that the pumps need not be interlocked, the dilution ratio need not be fixed, and any number of different methods may be used to select, control, and adjust the dilution ratio as desired.

The dilute composition and water are combined and passed through the static mixer 158 to provide for thorough mixing, reducing the risk of concentration spikes as the liquid passes the spectrophotometer 160. The spectrophotometer 160 senses the concentration of the antimicrobial in the passing liquid. It is preferred to use a multi-channel spectrophotometer so that adjustments may be made based upon regularly changing levels of background distortion. A spectrophotometer works well in accurately determining the concentration of an antimicrobial such as CPC in pure water and in accurately determining the concentration of CPC in a liquid having known, constant background characteristics. In the present system, the composition of and therefore background signal of the liquid being tested or measured is constantly changing, based upon changing amounts of fat, blood, and other organics, particles, and impurities. Because the background signal is unpredictable and constantly changing, it would be undesirable to use a fixed value for that background signal. Accordingly, a multi-channel spectrophotometer is used so that a value for the background signal may be determined with each reading. For example, a reading at a channel of approximately 254 nm to approximately 260 nm should provide an accurate reading for determining the amount of CPC and background material in a liquid, and a reading at a channel of approximately 280 nm should provide an accurate reading for determining the amount of background material in that liquid. Taking both readings allows an accurate background reading to be subtracted and therefore allows an accurate determination of the concentration of CPC in the liquid. It is undesirable to route the highly diluted liquid that passes the sensor 160 back into the recycle tank 92, so it is routed via line 162 to the capture tank 132.

The sensor 160 is operably connected to the controller 95. Accordingly, if the sensor 160 detects that the concentration of antimicrobial falls below a desired amount, the controller 95 may direct the chemical feed pump 93 to add more of the concentrated antimicrobial solution to bring the concentration of the antimicrobial in the dilute antimicrobial solution back up to the desired level. The system 10 can be configured to allow the potable water to be controlled in this fashion as well, but it is unlikely that there will be a need to add make-up water. In the preferred embodiment, the controller “learns” from the concentration measurements to adjust the rate at which CPC is introduced into the system to stay closer to a desired concentration. In usual operation, the controller is programmed to activate the pump 93 to provide a fixed amount of additional concentrate solution after a selected number of birds 20 are sprayed, with the volume of concentrate and the number of birds used being selected to approximate the amount of solution that will be carried away on the treated birds. With each concentration reading, for example every six minutes, the controller compares the measured CPC concentration with the target CPC concentration and calculates the percent deviation from the target. If the measured concentration is below the target concentration, the controller will decrease the selected number of birds used to determine the interval by a percent equal to the percent deviation below the target value. For example, if the system was adding concentrate antimicrobial solution after every 60^{th} bird, and the controller 95 determined that the measured CPC concentration was 10% below the target CPC concentration, the controller 95 would adjust the interval so that the concentrate CPC solution would be added after every 54^{th} bird. Similarly, if the concentration is above the target concentration, the controller will adjust the interval by increasing the selected number of birds by a percent equal to the percent deviation above the target value. For example, if the system was adding concentrate antimicrobial solution after every 70^{th} bird, and the controller 95 determined that the measured CPC concentration was 10% above the target CPC concentration, the controller 95 would adjust the interval so that the concentrate CPC solution would be added after every 77^{th} bird. This allows the system to automatically adjust or tune to changes, such as changes in line speed or bird weights. It typically takes approximately 4 to 6 samples for the system to adjust so that it is automatically adding the correct amount of make-up concentrate solution for maintaining the target CPC concentration for the new conditions.

In a somewhat similar control method, the flow rate to the manifolds 56, 58, 76, and 78 is continuously monitored. The controller 95 will typically go through 2 or 3 measuring loops before making any adjustments to the modulating valve 120 or 122, providing approximately a 5 second feedback delay. This reduces unnecessary adjustments and reduces the risk that a momentary aberrant reading will result in incorrect adjustments. To provide additional safety against over-application, the controller 95 is provided with upper and lower limits. If a flow rate reading is above or below the upper or lower limit, the controller 95 will immediately make the needed adjustment to modulating valve 120 or 122 without waiting for the regular 2 or 3 loop cycle.

At the end of the spray cycle, such as at the end of a shift or a day or other chosen period of time, the valve 126 is actuated to divert the dilute antimicrobial solution received from the recycle tank 92 and line 118 through line 130 to the capture tank 132. The pump 112 empties the recycle tank 92 and passes the dilute antimicrobial solution to the capture tank 132. Liquid is accumulated in the capture tank 132, until the liquid reaches a desired level. When the liquid in the capture tank 132 reaches the desired level, pump 168 empties the capture tank 132, passing the liquid through conduits 167 and through the series of drums 166 having carbon filters. The carbon in the drums 166 captures the antimicrobial to selectively remove the antimicrobial from the solution. In the preferred embodiment, three drums 166 are provided in series. The first drum 166 will capture most of the CPC, and the second drum 166 should capture the remaining CPC. Samples are taken periodically of the liquid passing from the second drum 166 to the third drum 166. If CPC is found above a desired trace level in the liquid passing from the second drum 166 to the third drum 166, the first drum 166 is sent for disposal or recycle, the second drum 166 is moved to the position previously occupied by the first drum 166, the third drum 166 is moved to the position previously occupied by the second drum 166, and a new drum 166 is moved to the position previously occupied by the third drum 166.

After passing through drums 166, the remaining, relatively antimicrobial-free liquid exits via line 170 and is disposed of in an appropriate manner, such as by being drained into a wastewater system of a plant. The frequency with which the system 10 will need to be purged will depend upon any number of factors, such as the number of work pieces 20 to be processed by the antimicrobial application unit 12 and the volume of the dilute antimicrobial solution required to charge the system 10 at the beginning of a spray cycle. A periodic purge of the system 10 will typically be used.

Other modifications, changes and substitutions are intended in the foregoing, and in some instances, some features of the invention will be employed without a corresponding use of other features. For example, the different features of the alternate embodiments may be merged or combined in any number of different combinations. Also, the antimicrobial application unit 12 may take any number of forms, shapes, and sizes and need not be one of the spray cabinet embodiments disclosed in U.S. Pat. No. 6,742,720 or PCT Application Serial No. PCT/US03/35933. Similarly, any number of different compositions may be used in any number of different concentrations, and the compositions may or may not include one or more antimicrobials. Further, any number of different separation techniques may be used in the antimicrobial separation unit, and the antimicrobial separation unit 15 may be used with or without a corresponding use of a capture tank 132. Further still, additional pumps, filters, and similar components may be incorporated into the system 10. Also, any number of different methods may be used to monitor the composition of the solution in the recycle tank 92. Similarly, the composition may be monitored constantly or at desired intervals. Further still, the drip tray 22 may not be used and may be any number of different lengths. Of course, quantitative information is included by way of example only and is not intended as a limitation as to the scope of the invention. Accordingly, it is appropriate that the invention be construed broadly and in a manner consistent with the scope of the invention disclosed.

What is claimed is:

1. A combination, comprising:
a housing;
a plurality of work pieces passing through said housing;
a first conduit disposed within said housing for applying a liquid to said work pieces;
a tank having an upper inlet and a lower inlet;
a second conduit connecting said housing to said upper inlet of said tank; and
a removable insert disposed within said tank, said insert comprising:
a rigid member, and
a helical member affixed to said rigid member, said removable insert being disposed within said tank so that said helical member is retained above said lower inlet.

2. The combination of claim 1, wherein said removable insert is disposed within said tank so that said helical member is retained below said upper inlet.

3. The combination of claim 1, further comprising an antimicrobial in said tank.

4. The combination of claim 3, wherein said upper inlet is disposed to provide for substantially tangential introduction of said antimicrobial into said tank.

5. The combination of claim 1, wherein said removable insert further comprises:
a baffle affixed to said rigid member, said removable insert being disposed within said tank so that said baffle is retained below said lower inlet.

6. The combination of claim 3, wherein said antimicrobial is present in said tank at a level that is above said lower inlet and below said upper inlet.

7. The combination of claim 1, wherein over at least 80 percent of a length of said second conduit, said second conduit is disposed to provide a vertical drop that is less than or equal to approximately one foot per foot of said second conduit.

8. The combination of claim 3, wherein over at least 80 percent of a length of said second conduit, said second conduit is disposed to provide a vertical drop that is less than or equal to approximately one quarter of an inch per foot of said second conduit.
